(19) United States
(12) Patent Application Publication
(10) Pub. No.: US 2016/0279073 A1
(43) Pub. Date: Sep. 29, 2016

(54) TERPENE AND CANNABINOID FORMULATIONS

(71) Applicant: FULL SPECTRUM LABORATORIES, LTD., Dublin 2 (IE)

(72) Inventors: Marc Donsky, Denver, CO (US); Robert Winnicki, Denver, CO (US)

(21) Appl. No.: 15/033,023

(22) PCT Filed: Oct. 31, 2014
(86) PCT No.: PCT/IB2014/003156

§ 371 Date: Apr. 28, 2016

Related U.S. Application Data
(60) Provisional application No. 61/898,024, filed on Oct. 31, 2013.

(51) Int. Cl. A61K 31/01; A61K 31/015; A61K 36/185; A61K 31/352; A61K 31/05; A61K 47/36; A61K 9/127; A61K 31/045

(52) U.S. Cl. CPC A61K 31/01; A61K 9/127; A61K 31/015; A61K 31/045; A61K 31/352; A61K 31/05; A61K 47/36; A61K 36/185

(57) ABSTRACT

The present invention provides stable, fast-acting liposome and micelle formulations of terpenes, hemp oil, cannabinoids, or mixtures of a cannabinoid and terpenes or hemp oil and cannabinoids that are suitable for pharmaceutical and nutraceutical applications. Also provided are methods for the manufacture of micelle and liposomal formulations.

### PRIOR APPLICATION INFORMATION

The instant application claims the benefit of U.S. Provisional Patent Application 61/898,024, filed on Oct. 31, 2013.

### FIELD OF THE INVENTION

The present invention relates to liposomal formulations of one or more terpenes, as well as a liposomal or a micellar formulation of one or more terpenes in combination with one or more cannabinoids or an analog of a cannabinoid. More specifically, the present invention relates to the manufacture and use of such formulations for medical, pharmaceutical and nutraceutical applications.

### BACKGROUND OF THE INVENTION

Terpenes and terpenoids are natural volatile non-aromatic compounds found as components of essential oils present in many plants and contain a carbon and hydrogen (terpenes) or a carbon, hydrogen, and oxygen scaffold (terpenoids). Terpenes and terpenoids have been used as fragrances and flavoring agents, as well as skin penetration agents. Terpenes are classified by the number of isoprene repeat units present in their molecular structure. Thus, hemiterpenes represent a class of compounds which consist of a single isoprene unit. Monoterpenes, such as geraniol, limonene and terpineol, consists of two isoprene units and conform to a molecular formula C₁₀H₁₆. Sesquiterpenes contain three isoprene units and have a molecular formula C₁₅H₂₄. Illustrative of this class are humulene, farnesene and farnesol.

Diterpenes, such as cafestol, kahweol, cembrene and taxadiene (a precursor of taxol), have four isoprene repeat units while sesterterpenes, such as geranylfarnesol, contain five isoprene units.

Triterpenes containing six isoprene units are the structural precursors of steroids. For instance, the triterpene squalene is a major constituent of shark liver oil that is used in the manufacture of lanosterol or cycloartenol. Sesquarterpenes, such as ferrugicadiol andtetraprenylcurcumene, contain seven isoprene units while tetraterpenes, such as lycopene, monocyclic gamma-carotene, and alpha- and beta-carotenes contain eight isoprene units. Terpenes having more than eight isoprene repeat units are referred to as polyterpenes. Rubber, for instance, is a polyterpene which consists of long chains of repeating isoprene units.

More than 120 different types of terpenes have been identified in extracts obtained from plants belonging to the genus Cannabis. The concentration of each terpene, however, has been shown to vary between the different plant cultivars.

Terpenes have poor solubility in water, but are readily soluble in non-aqueous and/or hydrophobic medium. Because of their lipophilic nature, terpenes can easily cross the blood-brain barrier and interact with cell membranes by binding to membrane receptors.

Cannabinoids are compounds derived from Cannabis sativa, an annual plant in the Cannabaceae family. The plant contains about 60 cannabinoids. The most active naturally occurring cannabinoid is tetrahydrocannabinol (THC), which is used for the treatment of a wide range of medical conditions, including glaucoma, AIDS wasting, neuropathic pain, treatment of spasticity associated with multiple sclerosis, fibromyalgia and chemotherapy-induced nausea. Additionally, THC has been reported to exhibit a therapeutic effect in the treatment of allergies, inflammation, infection, epilepsy, depression, migraine, bipolar disorders, anxiety disorder, and drug dependency and withdrawal syndromes. THC is particularly effective as an anti-emetic drug and is administered to curb emesis, a common side effect accompanying the use of opioid analgesics and anaesthetics, highly active anti-retroviral therapy and cancer chemotherapy.

Like terpenes, cannabinoids are lipophilic and potentially acid-labile compounds. Because of their hydrophobic nature, cannabinoids are poorly absorbed systemically from oral dosage forms in the aqueous environment of the gastrointestinal tract, and oral formulations of cannabinoids, therefore, exhibit low bioavailability.

The present invention overcomes the drawbacks described above by providing a stable liposomal composition of a terpene as well as stable liposomal and micellar compositions of a terpene-cannabinoid mixture. The inventive compositions are suitable for medical, pharmaceutical and nutraceutical applications. In addition, the present invention provides stable liposomal compositions of hemp oil as well as stable liposomal and micellar compositions that contain a mixture of hemp oil and cannabinoid. The formulations according to the present invention are suitable for medical, pharmaceutical and nutraceutical applications. Also described is the use of such formulations for stimulating intellectual activity and mental concentration, and to procure calming effects (Tambe Y et al., 1996, Planta Med. 62(5):469-70; Tembaro and Bartolato 2012, Recent Pat CNS Drug Discovery 7(1) 25-40).

### SUMMARY OF THE INVENTION

It is therefore an aspect of the invention to provide solutions to the aforementioned deficiencies in the art. To this end the invention provides a stable liposomal formulation of a primary, secondary or tertiary terpene in an aqueous solution. Illustrative of the class primary terpene are α-pinene, α-bisabolol, β-pinene, guaiene, guaiol, limonene, myrcene or ocimene. The amount of the primary terpene in the inventive formulation is 50% (w/w), while the amount of the secondary terpene is from about 30% to about 40% (w/w) and the amount of the tertiary terpene is from about 8% to about 10% (w/w) of the formulation.

Each unique cultivar of Cannabis sativa may have a unique cannabinoid and cannabinoid-terpene profile. This invention describes Primary, Secondary and Tertiary terpene groups. Terpenes can be extracted, collected and encapsulated in liposomes or micelles. There is a growing body of research on the sedative and stimulatory properties of terpenes, and on the organoprotective, anti-cancer and anti-viral effects of individual and combinations of terpenes and essential oils. Certain terpenes are known to have medicinal properties that have been utilized in aromatherapy, as well as for sedative, analgesic, anti-inflammatory, antibiotic, anti-fungal and mental stimulation. This invention includes unique groupings that have been observed in various cultivars of Cannabis sativa that may have medicinal value.

In one embodiment, the stable inventive liposomal suspensions can further comprise one or more cannabinoids or cannabinoid analogs. The average diameters of liposomes in a formulation according to the invention is in a range between 50 nm to 1000 nm and the final maximum concentration of cannabinoids or cannabinoid analogues in the above described formulation is from 0.01 g/liter to 100 g/liter. Any cannabinoids or cannabinoid analogues selected from natural compounds, synthetic compounds, semi-synthetic compounds, or mixtures thereof can be used in the claimed suspension. Exemplary compounds within the class cannabinoids or cannabinoid analogues include without limitation cannabinol, cannabidiol, Δ9-tetrahydrocannabinol, Δ-8-tetrahydrocannabinol, 11-hydroxy-tetrahydrocannabinol, 11-hydroxy-Δ-9-tetrahydrocannabinol, levonantradol, Δ-11-tetrahydrocannabinol, tetrahydrocannabivarin, dronabinol, amandamide, nabilone, a combination thereof, a synthetic analogue of a natural cannabinoid, as well as compounds having a basic cannabinoid structure and having physiological properties similar to natural or synthetic cannabinoids.

For certain embodiments, the liposomal suspension can further comprise a stabilizer. The stabilizer may be guar gum, xyanthan gum, cellulose, hyaluronic acid, polyvinyl pyrrolidone (PVP), alginate, chondritin sulfate, polygamma glutamic acid, gelatin, chitisin, corn starch or flour and is present in an amount from about 0.1% to about 2% (w/v).

In one aspect of the invention, the primary terpene in the suspension is α-pinene, the secondary terpene is myrcene, β-pinene or t-carophyllene, and the tertiary terpene is β-pinene, t-carophyllene, α-bisabolol or myrcene. Such a suspension may also contain trace amounts of one or more other terpenes selected from the group consisting of α-humulene, α-bisabolol, guaiene, limonene, ocimene, terpinolene, 3-carene, myercene, guaiol, α-terpineol and linalool.

For certain suspensions, the primary terpene in the suspension is α-bisabolol, the secondary terpene is t-carophyllene, and the tertiary terpene is α-pinene or myrcene and the suspension can further comprise trace amounts of one or more terpenes selected from the group consisting of α-humulene, α-terpineol guaiol, and linalool.

Alternatively, the invention provides a suspension comprising β-pinene as the primary terpene, α-pinene as the secondary terpene and t-carophyllene or terpinolene as the tertiary terpenes. Such a suspension can further comprise trace amounts of myrcene.

Illustrative of other liposomal suspensions are the following:

(a) suspension in which the primary terpene is guaiene, the secondary terpene is t-carophyllene; and the tertiary terpenes are myrcene or a humulene with trace amounts of α-pinene, α-bisabolol, β-pinene, limonene, ocimene and/or terpinolene;

(b) suspension in which the primary terpene is guaiol, the secondary terpene is α-bisabolol, and the tertiary terpene is t-carophyllene or myrcene with trace amounts of α-pinene, α-terpineol, α-humulene and terpinolene.

(c) suspension in which the primary terpene is limonene, the secondary terpenes are myrcene or t-carophyllene; and the tertiary terpenes are selected from the group consisting of linalool, myrcene, β-pinene and t-carophyllene, α-bisabolol and myrcene. The suspension may further comprise trace amounts of one or more terpenes selected from α-humulene, α-pinene, β-pinene, fenchol, guaiene, linalool, ocimene or α-terpineol.

(d) suspension in which the primary terpene is myrcene, the secondary terpene is selected from the group consisting of α-pinene, t-carophyllene, terpinolene, ocimene, limonene and linalool and the tertiary terpene is selected from the group consisting of β-pinene, t-carophyllene, limonene, ocimene, myrcene, α-pinene, bisabolol and myrcene. The suspension may further comprise trace amounts of one or more terpenes selected from the group consisting of α-humulene, α-bisabolol, guaiene, limonene, ocimene, 3-carene, β-pinene, α-pinene, myercene, guaiol, α-terpineol, terpinolene and linalool.

The invention also provides a stable, aqueous, liposomal suspension comprising hemp oil and one or more cannabinoids or cannabinoid analogues. Preferably, the cannabinoids or cannabinoid analogues in the inventive suspensions are natural compounds, synthetic compounds, semi-synthetic compounds, or mixtures thereof. Illustrative of such compounds are cannabinol, cannabidiol, Δ-9-tetrahydrocannabinol, Δ-8-tetrahydrocannabinol, 11-hydroxy-tetrahydrocannabinol, 11-hydroxy-Δ-9-tetrahydrocannabinol, levonantradol, Δ-11-tetrahydrocannabinol, tetrahydrocannabivarin, dronabinol, amandamide, nabilone, a combination two or more such compounds, or a mixture of a natural cannabinoid and a synthetic analogue of a cannabinoid. The average size of the micelle or liposome in the aqueous suspension can range between 50 nm and 1000 nm and the final maximum concentration of cannabinoids or cannabinoid analogues in the inventive formulation is from 0.01 g/liter to 100 g/liter.

Suspensions according to the present invention can further include one or more stabilizers in an amount from about 0.1% to about 2% (w/v). Exemplary of such stabilizing agents include without limitation guar gum, xyanthan gum, cellulose, hyaluronic acid, polyvinyl pyrrolidone (PVP), alginate, chondritin sulfate, poly gamma glutamic acid, gelatin, chitisin, corn starch and/or flour.

Also provided is a method for producing a stable, highly concentrated liposomal formulation of one or more terpenes by: (a) dissolving one or more terpenes in ethanol to obtain an ethanolic solution of the terpene; (b) adding a phospholipid to the ethanolic solution of terpenes; (c) injecting the ethanolic solution of phospholipid and terpenes into distilled water to obtain a liposomal suspension of terpenes; and (d) removing the ethanol from the liposomal suspension of terpenes to obtain a stable liposomal suspension of one or more terpenes. The final maximum concentration of terpenes in the liposomal suspension described above is from about 0.001 gm/L to about 100 g/liter.

In an exemplary composition, the hydrophobic/lipophilic membrane of the liposome can comprise about 40% phosphatidylcholine, about 3.5% phosphatidylethanolamine, about 6% phosphonophospholipids, and about 0.5% of other phospholipids. According to another exemplary composition the hydrophobic/lipophilic membrane of liposomes in the inventive composition can comprise about 26% phosphatidylcholine, about 10% phosphatidylethanolamine, about 13% phosphonophospholipids, and about 1% of other phospholipids.

In one aspect, the method for producing a liposomal suspension thus described is qualified in that one or more cannabinoids or cannabinoid analogues are dissolved in the ethanolic solution of one or more terpenes to obtain terpene-cannabinoid liposomes. Preferably, the cannabinoids or cannabinoid analogs are selected from the group consisting of cannabinol, cannabidiol, Δ-9-tetrahydrocannabinol, Δ-8-tetrahydrocannabinol, 11-hydroxy-tetrahydrocannabinol, 11-hydroxy-Δ-9-tetrahydrocannabinol, levonantradol, Δ11-tetrahydrocannabinol, tetrahydrocannabivarin, dronabinol, amandamide, nabilone, and a combination of two or more of these compounds. Preferably, the average diameter of the liposomes in the suspension is in a range between 50 nm and 1000 nm, and the final maximum concentration of cannabinoids or cannabinoid analogues in the formulation is from about 0.01 g/liter to about 100 g/liter.

In yet another embodiment, the invention provides a method of producing a stable, highly concentrated liposomal formulation of hemp oil that comprises the steps of: (a) dissolving hemp oil in ethanol to obtain an ethanol hemp oil solution; (b) adding a phospholipid to the ethanol hemp oil solution to obtain an ethanol-phospholipid hemp oil solution; (c) injecting the ethanol-phospholipid hemp oil solution into distilled water to obtain a liposomal hemp oil suspension; and (d) removing the ethanol from the liposomal hemp oil suspension, thereby producing a stable liposomal suspension of hemp oil. Preferably, the final maximum concentration of hemp oil in the liposomal suspension is from about 0.01 g/L to about 200 g/L.

In one aspect of the invention, the method further comprises dissolving one or more cannabinoids or cannabinoid analogues in an ethanolic solution of hemp oil to obtain an ethanolic hemp oil/cannabinoid solution. To obtain liposomes, a phospholipid is added to the ethanolic hemp oil/cannabinoid solution followed by injection of the phospholipid-hemp oil-cannabinoid solution into distilled water to obtain a suspension of hemp oil-cannabinoid liposomes. Removal of ethanol from this suspension results in a stable concentrated suspension containing hemp oil-cannabinoid liposomes. Preferably, the cannabinoids or cannabinoid analogs are selected from the group consisting of cannabinol, cannabidiol, Δ-9-tetrahydrocannabinol, Δ-8-tetrahydrocannabinol, 11-hydroxy-tetrahydrocannabinol, 11-hydroxy-Δ-9-tetrahydrocannabinol, levonantradol, Δ-11-tetrahydrocannabinol, tetrahydrocannabivarin, dronabinol, amandamide, nabilone, a combination thereof, a natural or synthetic analogue thereof, and a natural or synthetic molecule with a basic cannabinoid structure. In a preferred aspect of the invention, the average liposome diameter size in the suspension is in a range between 50 and 1000 nm, and the final maximum concentration of cannabinoids or cannabinoid analogues in the liposome suspension is from 0.01 g/liter to 100 g/liter.

In another embodiment, the invention provides a method to produce a stable formulation of hemp oil and cannabinoids that includes the steps of: (a) dissolving cannabinoid oil in ethanol to obtain an ethanol-cannabinoid solution; (b) injecting the ethanol-cannabinoid solution in distilled water to obtain an ethanol-cannabinoid emulsion; (d) adding a carbohydrate, such as glycerol, to the ethanol-cannabinoid emulsion and blending this mixture to obtain ethanol-cannabinoid-carbohydrate solution; (e) adding lecithin with terpenes and hemp oil to the above mixture and blending to obtain a stable cannabinoid-terpene-hemp oil formulation. The concentration range of cannabinoids or cannabinoid analogues in the inventive formulation is from 0.01 g/liter to 100 g/liter. The final maximum concentration of terpenes in the liposomal suspension described above is from about 0.001 gm/L to about 100 g/liter.

In an additional embodiment, the methods for producing stable, highly concentrated, inventive liposomal formulations of a terpene/cannabinoid or a hemp oil-cannabinoid may further comprise the steps of: (e) adding sodium alginate to the liposomal suspension in a final concentration of 2% to obtain an alginate liposomal terpene/cannabinoid suspension or an alginate liposomal hemp oil-cannabinoid suspension; (f) adding the alginate liposomal terpene/cannabinoid or a hemp oil-cannabinoid suspension to a calcium chloride solution to obtain a calcium alginate-encapsulated liposomal terpene/cannabinoid suspension or an alginate-encapsulated liposomal hemp oil-cannabinoid suspension; (g) cold-pressing, air-drying, spray drying or freeze drying the calcium alginate-encapsulated liposomal terpene/cannabinoid or hemp oil-cannabinoid suspensions to remove the water and obtain a dry terpene/cannabinoid or hemp oil-cannabinoid powder; and (h) re-suspending the dry powder in citrate buffer to obtain an aqueous terpene/cannabinoid or hemp oil-cannabinoid solution. Preferably, the amount of cannabinoid or cannabinoid analogue in the aqueous terpene/cannabinoid solution or a hemp oil-cannabinoid solution is 10% w/w to 80% w/w. The amount of hemp oil in the hemp oil-cannabinoid solution is 10% w/w to 80% w/w, the amount of terpene in the aqueous terpene-cannabinoid solution is 1% w/w to 10% w/w and the phospholipid content can range from about 20% to about 99%.

In an alternative embodiment, the methods of producing stable, highly concentrated, inventive liposomal formulations of a terpene/cannabinoid or a hemp oil-cannabinoid may further comprise the steps of: (e) adding sodium alginate to the liposomal terpene/cannabinoid or hemp oil-cannabinoid suspension in a final concentration of 4% to obtain an alginate liposomal terpene/cannabinoid or hemp oil-cannabinoid suspension; (f) pouring the terpene/cannabinoid or hemp oil-cannabinoid liposomal suspensions into flat trays to a depth of 0.5 cm; (g) further desiccating the suspensions at 50° C. for 24 hours to yield a 1 mm film; and (h) dissolving the film in distilled water to obtain a terpene/cannabinoid aqueous solution or a hemp oil-cannabinoid suspension. Preferably, the amount of cannabinoid or cannabinoid analogue in the aqueous terpene/cannabinoid solution or a hemp oil-cannabinoid solution is from about 10% w/w to about 80% w/w. The amount of hemp oil in the hemp oil-cannabinoid solution is from about 10% w/w to about 80% w/w, the amount of terpene in the aqueous terpene-cannabinoid solution is from about 1% w/w to about 10% w/w and the phospholipid content can range from about 20% to about 99%.

In yet another alternative embodiment, the methods of producing stable, highly concentrated liposomal formulations of a terpene/cannabinoid or a hemp oil-cannabinoid may further comprise the steps of: (e) adding L-leucine and a sugar selected from the group consisting of lactose and sucrose to the liposomal terpene/cannabinoid or hemp oil-cannabinoid suspension to obtain a sugar liposomal terpene/cannabinoid or hemp oil-cannabinoid suspension; (f) spray-drying the sugar liposomal terpene/cannabinoid or hemp oil-cannabinoid suspension at 55° C. to remove the water so as to obtain a dry terpene/cannabinoid or hemp oil-cannabinoid powder; (g) milling the dry terpene/cannabinoid or hemp oil-cannabinoid powder and re-suspending the dry powder in water to obtain an aqueous terpene/cannabinoid solution or an aqueous hemp oil-cannabinoid solution. The amount of cannabinoid or cannabinoid analogue in the aqueous terpene/cannabinoid solution is from about 10% w/w to about 80% w/w. The amount of terpene in the aqueous terpene-cannabinoid solution is from about 1% w/w to about 10% w/w and the phospholipid content can range from about 20% to about 99%.

In a different embodiment, the invention provides an aqueous terpene/cannabinoid solution comprising a primary terpene, a secondary terpene and a tertiary terpene, and one or more cannabinoids or cannabinoid analogues. Preferably the terpenes and cannabinoids or cannabinoid analogs in the solution are in an amount of 50 g/liter. In a preferred aspect of the invention, the solution is in the form of a fast-acting pharmaceutical composition, a nutraceutical composition, or a food or beverage for administration to a subject. Even more preferably, the pharmaceutical composition and the nutraceutical composition are fast-acting formulations for oral, enteral, parenteral, intravenous, pulmonary, mucosal, sub-mucosal or topical administration.

In another embodiment, the invention provides an aqueous solution of hemp oil and a cannabinoid comprising hemp oil and one or more cannabinoids or cannabinoid analogues. Preferably, the concentration of hemp oil and one or more cannabinoids or cannabinoid analogs in the solution is about 50 g/liter. In a preferred aspect, the solution is in form of a fast-acting pharmaceutical composition, a nutraceutical composition, or a food or beverage for administration to a subject. Even more preferably, the pharmaceutical composition and the nutraceutical composition are fast-acting formulations for oral, enteral, parenteral, intravenous, pulmonary, mucosal, sub-mucosal or topical administration.

Other advantages, and novel features will be readily apparent to those skilled in the art from the following detailed description of the invention.

### DETAILED DESCRIPTION OF THE PREFERRED EMBODIMENTS

The present invention provides stable, fast-acting formulations of terpenes, a mixture of terpenes and cannabinoids or cannabinoid analogs, hemp oil, or a mixture of hemp oil and cannabinoids or cannabinoid analogs. The term “analog” refers to a compound that is structurally related to naturally occurring cannabinoids, but whose chemical and biological properties may differ from naturally occurring cannabinoids. In the present context, analog or analogs refer to compounds that may not exhibit one or more unwanted side effects of a naturally occurring cannabinoid. Analog also refers to a compound that is derived from a naturally occurring cannabinoid by chemical, biological or a semi-synthetic transformation of the naturally occurring cannabinoid.

According to one aspect, there are provided aqueous compositions comprising one or more terpenes, a mixture of terpenes and cannabinoids or a mixture of terpenes and a cannabinoid analog. The inventive formulations also encompass formulations of hemp oil, or a mixture of hemp oil and cannabinoids or cannabinoid analogs. Because terpenes, hemp oil and cannabinoids are hydrophobic in nature, stable colloidal formulations can be obtained by contacting a solution containing any one of the above mentioned components with a solvent such as water, with or without pharmaceutically acceptable buffers. Other solvents suitable for forming colloids include C₁-C₆ aliphatic alcohols or mixtures of water and C₁-C₆ aliphatic alcohols, acetone, mixtures of water and acetone, or any water miscible organic solvent.

In one of its aspects, the inventive formulations are in the form of liposomes that encapsulate one or more terpenes, a mixture of one or more terpenes and a cannabinoid or a cannabinoid analog. The inventive formulations in another embodiment comprise liposomes containing hemp oil, or a mixture of hemp oil and a cannabinoid or a cannabinoid analog. Compositions of cannabinoids or an analog of a cannabinoid also can be formulated as a micelle. Within the context of the present technology, the term “micelle” refers to an aggregate of surfactant molecules dispersed in a liquid colloid, while “liposome” refers to a vesicle composed of a lipid monolayer or bilayer. Additionally, mixtures of one or more terpenes and a cannabinoid or a mixture of one or more terpenes and a cannabinoid analog can be formulated as a micelle. The concentration of the terpene in such a micelle is the range from about 0.001 g/L to about 5 g/L while the concentration of the cannabinoid or the cannabinoid analog can range from about 0.01 g/L to about 5 g/L.

Other drugs as well as pharmaceutically acceptable carriers may also be present in the inventive formulations. These additional components, if present in the inventive suspension, can be associated with the lipophilic membrane of a liposome or can be entrapped in the aqueous fluid that forms the core of the liposome. The entrapped terpenes, mixtures of terpenes and cannabinoids, mixtures of terpenes and a cannabinoid analog, hemp oil, mixture of hemp oil and cannabinoids or mixture of hemp oil and cannabinoid analogs can contribute to the stability of the micelle/liposome membranes, permitting the use of such formulations as improved, fast-acting, reliable and efficient systems for the oral, enteral, parenteral, intravenous or topical delivery of the above mentioned components. The term “subject” refers to a mammal in need of treatment or undergoing treatment using the inventive compositions or desirous of being administered the inventive compositions. Mammalian subjects include without limitation humans, dogs, cats, horses or any other animal in need of treatment. Thus, the inventive compositions can be used for human and veterinary purposes.

The inventive suspensions comprising one or more terpenes, a mixture of a terpene and cannabinoid or cannabinoid analog, hemp oil, mixture of hemp oil and cannabinoid or mixture of hemp oil and cannabinoid analogs can be manufactured as thermostable unilamellar micelles or liposomes. Such micelles or liposomes are stable at temperatures greater than 50° C. and are obtained by rapidly dissolving or injecting through a small orifice under pressure a solution of any of the above mentioned components into one of the above described aqueous solvents or an aqueous solution of a pharmaceutically active compound or drug. Alternatively, ultrasonic atomization can be used to form the inventive formulations.

In one of its aspects, the inventive micellar or liposomal compositions can further comprise a stabilizing agent. Illustrative of such stabilizing agents include polymers or compounds selected from the group consisting of cellulose hyaluronic acid, polyvinyl pyrrolidone (PVP), alginate, chondroitin sulfate, poly gamma glutamic acid, gelatin, chitisin, corn starch and flour.

The size of micelles in a formulation containing a cannabinoid, a cannabinoid analog, or micelle formulations containing a mixture of a terpene and a cannabinoid or cannabinoid analog, is from about 0.01 μm to about 2.0 μm. For certain embodiments, the size of the spherical micelles is about 0.05 μm, about 0.1 μm, about 0.15 μm, 0.2 μm, 0.25 μm, 0.3 μm, 0.35 μm, 0.4 μm, 0.45 μm, 0.5 μm, 0.55 μm, 0.6 μm, 0.7 μm, 0.75 μm, 0.8 μm, 0.85 μm, 0.9 μm, about 0.95 μm, about 1.0 μm, 1.20 μm, 1.40 μm, 1.50 μm, 1.60 μm, 1.70 μm, 1.80 μm, 1.90 μm and 2.0 μm. For certain embodiments, micelles that are about 0.04 μm, about 0.05 μm, about 0.06 μm, about 0.07 μm, about 0.08 μm, or about 0.09 μm are used to formulate the inventive compositions.

According to one aspect, the present invention provides a micelle that comprises a terpene, a mixture of a terpene and a cannabinoid as well as a method for the manufacture of such a micelle formulation. According to the inventive methodology, a stable aqueous micelle formulation of a terpene and a cannabinoid or a terpene and a cannabinoid analog can be obtained by dissolving the terpene and the cannabinoids or a cannabinoid analog in ethanol to obtain an ethanolic solution containing a mixture of a terpene and a cannabinoid or a cannabinoid analog. This ethanolic solution is then injected into distilled water to obtain an aqueous-ethanolic suspension of micelles. To obtain the desired final formulation, the aqueous-ethanolic suspension of micelles is concentrated using rotary evaporation to remove the ethanol, to produce a stable aqueous micelle formulation. The inventive micelle formulations do not contain phospholipids or cholesterol and shows no aqueous core when observed under oil immersion microscopy.

Any natural or synthetic cannabinoid can be used to manufacture the above described micelles. Illustrative of such compounds include without limitation cannabinoids or cannabinoid analogues selected from the group consisting of cannabinol, cannabidiol, Δ9-tetrahydrocannabinol, Δ8-tetrahydrocannabinol, 11-hydroxy-tetrahydrocannabinol, 11-hydroxy-Δ9-tetrahydrocannabinol, levonantradol, Δ11-tetrahydrocannabinol, tetrahydrocannabivarin, dronabinol, amandamide, nabilone, and a combination of two or more of these compounds.

The maximum final concentration of a cannabinoid or a cannabinoid analog in the micelle is in the range from about 0.1 g/L (0.1 mg/mL) to about 5 g/L (5 mg/mL). For certain embodiments, the concentration of a cannabinoid or a cannabinoid analog in the micelle of the inventive formulation is from about 1 g/L to about 4 g/L, from about 1 g/L to about 3 g/L, from about 1 g/L to about 2 g/L, or from about 1 g/L to about 1.5 g/L. The concentration of a cannabinoid or a cannabinoid analog in the inventive micelle formulation can be about 0.2 g/L, about 0.3 g/L, about 0.4 g/L, about 0.5 g/L, about 0.6 g/L, about 0.7 g/L, about 0.8 g/L, about 0.9 g/L, about 1.0 g/L, about 1.1 g/L, about 1.2 g/L, about 1.3 g/L, about 1.4 g/L, about 1.5 g/L, about 1.6 g/L, about 1.7 g/L, about 1.8 g/L, about 1.9 g/L, or about 2.0 g/L.

When the micelle contains a mixture of a terpene and a cannabinoid or a cannabinoid analog the concentration of the terpene is from about 0.001 g/L to about 0.5 g/L. Illustrative of micelle formulations comprising a mixture of a terpene and a cannabinoid or an analog of a cannabinoid are compositions in which the concentration of terpene is from about 0.001 g/L to about 0.4 g/L, from about 0.001 g/L to about 0.3 g/L, from about 0.001 g/L to about 0.2 g/L, from about 0.001 g/L to about 0.1 g/L. For certain formulations, the concentration of the terepene can range from about 0.1 g/L to about 0.5 g/L. For instance, the concentration of terpene in the inventive micelle formulation containing a mixture of terpene and cannabinoid or cannabinoid analog is about 0.002 g/L, about 0.003 g/L, about 0.004 g/L, about 0.005 g/L, about 0.006 g/L, about 0.007 g/L, about 0.008 g/L, about 0.009 g/L, about 0.01 g/L, about 0.02 g/L, about 0.03 g/L, about 0.04 g/L, about 0.05 g/L, about 0.06 g/L, about 0.07 g/L, about 0.08 g/L, about 0.09 g/L, about 0.1 g/L, 0.2 g/L, 0.3 g/L, or about 0.4 g/L. In some embodiments the ratio of cannabinoid to terpene in the micelle formulation is from about 5:1 to about 10:1.

Typical concentrations of a cannabinoid or a cannabinoid analog in a terpene/cannabinoid liposomal suspension is from about 2 g/L to about 50 g/L, although, maximum final concentration of cannabinoids or an analog of the cannabinoid in the liposomal formulation can be in the range from about 0.01 g/L to about 100 g/L. The concentration of terpene in such a liposomal composition can range from about 0.001 g/L to about 100 g/L. Illustrative liposome formulations containing a mixture of a terpene and a cannabinoid or a cannabinoid analog can contain from about 0.1 g/L to about 90 g/L, from about 0.1 g/L to about 80 g/L, from about 0.1 g/L to about 70 g/L, from about 0.1 g/L to about 60 g/L, from about 0.1 g/L to about 50 g/L, from about 0.1 g/L to about 40 g/L, from about 0.1 g/L to about 30 g/L, from about 0.1 g/L to about 20 g/L, or from about 0.1 g/L to about 10 g/L, of a cannabinoid or a cannabinoid analog.

Depending on the amount of cannabinoid or cannabinoid analog present in the liposome formulation, the amount of terpene in such a formulation can be from about 0.001 g/L (0.001 mg/mL) to 100 g/L (100 mg/mL). For certain formulations, the amount of terpene is from about 10 g/L to about 70 g/L. Illustrative of liposome formulations comprising a mixture of a terpene and a cannabinoid or an analog of a cannabinoid are compositions in which the concentration of terpene is from about 10 g/L to about 60 g/L, from about 10 g/L to about 50 g/L, from about 10 g/L to about 40 g/L, from about 10 g/L to about 30 g/L, from about 10 g/L to about 20 g/L.

The maximum concentration of terpenes and cannabinoids in the liposomes according to the present invention is about 100 g/L. Thus, the inventive liposome formulation can have a cannabinoid:terpene ratio of 0.1 g/L:99.9 g/L, 1 g/L:99 g/L, 5 g/L:95 g/L, 15 g/L:85 g/L, 25 g/L:75 g/L, 35 g/L:65 g/L, 45 g/L:55 g/L, 50 g/L:50 g/L, 55 g/L:45 g/L, 65 g/L:35 g/L, 75 g/L:25 g/L, 85 g/L:15 g/L, or 95 g/L:5 g/L. The size of the unilamellar spherical liposomes in the inventive composition described above are from about 0.1 μm to about 2.0 μm, for example, about 0.2 μm, 0.3 μm, 0.4 μm, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, or 1.9 μm.

Formulations pursuant to the present invention are particularly suitable for oral, enteral, parenteral, intravenous, pulmonary, mucosal, sub-mucosal or topical administration. The inventive formulations may be administered to subjects in need of treatment related to pain, allergies, inflammation, infection, epilepsy, depression, migraine, bipolar disorders, anxiety disorder, and drug dependency and withdrawal syndromes (Crowell and Gould, 1994, Crit Rev Oncog. 5(1):1-2; Cridge and Rosengren, 2013, Cancer Manag Res. 5:301-13; Salminen et al., 2008, Cellular and Molecular Life Sciences 65, pp 2979-2999; Nóbrega de Almeida et al., 2001, Molecules 16, 2726-2742; Smith, 2005, Curr Opin Investig Drugs 6(7): 680-5; Grotenhermen, 2004, Neuroendocrinology Letters Nos. 1/2, February-April Vol. 25; Jin et al., 2011, Archives of Pharmacal Research 34, pp 223-228; Cabral, 2001, Journal of Cannabis Therapeutics 1, pp 61-85; Blaas, 2008, Cannabinoids 3(2):8-10; Ashton et al., 2005, J Psycopharmacol. 19(3):293-300; Scavone et al., 2013, Neuroscience 248:637-54).

Any terpene may be used in the formulations of the invention. Preferred terpenes include, but are not limited to, α-pinene, myrcene, β-pinene, t-carophyllene, β-pinene, α-bisabolol, α-humulene, guaiene, limonene, ocimene, terpinolene, 3-carene, guaiol, α-terpineol, linalool, fenchol. Preferably, the formulations comprise a primary terpene, a secondary terpene, and a tertiary terpene, and may comprise trace amounts of additional terpenes. According to one embodiment, the amount of the primary terpene in the formulations is 50% (w/w), the amount of the secondary terpene is up to 40% (w/w) and the amount of the tertiary pinene is up to 10% (w/w) of the formulation. The terpenes may be isolated from any plant, including the Cannabis sativa plant. Many of the formulations according to the present invention can comprise hemp oil.

For inventive formulations comprising a cannabinoid and terpenes, the cannabinoid is selected from the group consisting of cannabinol, cannabidiol, Δ9-tetrahydrocannabinol, Δ8-tetrahydrocannabinol, 11-hydroxy-tetrahydrocannabinol, 11-hydroxy-Δ9-tetrahydrocannabinol, levonantradol, Δ-11-tetrahydrocannabinol, tetrahydrocannabivarin, dronabinol, amandamide, and nabilone. Moreover, any combination of two or more of the above mentioned cannabinoids can be present in the inventive formulations. The inventive formulations can also contain cannabimimetic agents. The phrase “cannabimimetic agent” refers to any substance that is a cannabinoid receptor agonist as demonstrated by binding studies and functional assays. For instance, a cannabimimetic agent can be a compound having: (i) 2-(3-hydroxycyclohexyl)phenol core that can be further substituted at the 5-position of the phenolic ring by alkyl or alkenyl group; (ii) 3-(1-naphthoyl)indole or 3-(1-naphthylmethane)indole core further substituted at the nitrogen atom of the indole ring, whether or not substituted on the naphthoyl or naphthyl ring to any extent; (iii) 3-(1-naphthoyl)pyrrole core further substituted at the nitrogen atom of the pyrrole ring, whether or not substituted on the naphthoyl ring to any extent; (iv) 1-(1-naphthylmethylene)indene core further substituted at the 3-position of the indene ring, whether or not substituted on the naphthyl ring to any extent; and (v) 3-phenylacetylindole or 3-benzoylindole core further substituted at the nitrogen atom of the indole ring, whether or not substituted on the phenyl ring to any extent.

Naturally occurring cannabinoids are compounds obtained from plant tissue, for example, the trichomes of the Cannabis sativa plant. The cannabinoids can be extracted from plant tissue by suspending the latter in an appropriate solvent to obtain a crude extract followed by analytical or preparative purification of such an extract to provide pharmaceutical grade cannabinoid compounds. Alternatively, cannabinoid compounds will be extracted from plant tissue under supercritical conditions. Solvents that can be used for supercritical extraction of cannabinoids include without limitation carbon dioxide or other gases in isolation or combination with or without solvent modifiers, selected from ethanol, propanol, butanol, hexane, chloroform, dichloromethane, acetone, or any organic solvent capable of extracting cannabinoids, and alcohol-water mixtures, for instance water-ethanol or water-butanol mixtures.

In addition to natural cannabinoids, the present technology encompasses synthetic cannabinoid compounds as well as cannabinoids that can be obtained using semi-synthetic protocols. The manufacture of cannabinoid compounds or an analog of a cannabinoid using a semi-synthetic protocol involves contacting an appropriate substrate with one of the cannabinoid synthase enzymes. For instance, tetrahydrocannabinolic acid (THCA) or its analogs can be manufactured semi-synthetically by contacting cannabigerolic acid (CBGA) or an appropriately substituted derivative of CBGA with THC synthase to obtain the corresponding THCA or THCA analog respectively.

Pursuant to the semi-synthetic strategy, a Formula I compound can be brought in contact with a cannabinoid synthase, for example, cannabidiolic acid synthase, a tetrahydrocannabinoilic acid synthase or a cannabichromene acid synthase to promote the enzyme catalyzed conversion of the Formula I compound to a cannabinoid or cannabinoid analog.

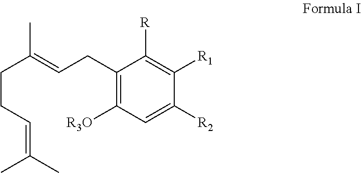

The structure and biochemical properties of products obtained via such a synthetic strategy will depend on (a) the cannabinoid synthase used and (b) the chemical identities of substituent groups R, R₁, R₂ and R₃ in the Formula I compound used.

Thus for Formula I compounds, R can be selected from —OH, halogen, —SH, or a NRₐR_{b} group. Substituents R₁ and R₂ are each independently selected from the group consisting of H, —C(O)Rₐ, —ORₐ, an optionally substituted C₁-C₁₀ linear or branched alkylene, an optionally substituted C₂-C₁₀ linear or branched alkenylene, an optionally substituted C₂-C₁₀ linear or branched alkynylene, an optionally substituted C₃-C₁₀ aryl, an optionally substituted C₃-C₁₀ cycloalkyl, (C₃-C₁₀)aryl-(C₁-C₁₀)alkylene, (C₃-C₁₀)aryl-(C₂-C₁₀)alkenylene, and (C₃-C₁₀)aryl-(C₁-C₁₀)alkynylene.

For certain Formula I compounds, R₁ and R₂ together with the carbon atoms to which they are bonded form a C₅-C₁₀ cyclic ring. In one aspect, the C₅-C₁₀ cyclic ring comprises one or more heteroatoms selected from oxygen, sulfur or nitrogen. R₃ in Formula I can be selected from the group consisting of H, —C(O)Rₐ and C₁-C₁₀ linear or branched alkyl, with groups Rₐ and R_{b} each independently being H, —OH, —SH, —NH₂, (C₁-C₁₀) linear or branched alkyl, or a C₃-C₁₀ cycloalkyl.

The inventive compositions have unexpected advantageous properties. For instance, liposomal compositions according to the present invention are stable at high temperatures exceeding 50° C. and also are stable to sonication. Further, the liposomal compositions according to the present invention are capable of carrying large payloads of terpenes, hemp oil, mixtures of terpenes and cannabinoids or mixtures of hemp oil and cannabinoids. The payload of the inventive liposome formulations can range from about 0.01 g/L to about 100 g/L. Exemplary liposome formulations can have payloads in the range from about 0.01 g/L to about 0.1 g/L, from about 0.01 g/L to about 0.5 g/L, from about 0.01 g/L to about 1 g/L, from about 0.01 g/L to about 5 g/L, from about 0.01 g/L to about 10 g/L, from about 0.01 g/L to about 20 g/L, from about 0.01 g/L to about 30 g/L, from about 0.01 g/L to about 40 g/L, from about 0.01 g/L to about 50 g/L, from about 0.01 g/L to about 60 g/L, from about 0.01 g/L to about 70 g/L, from about 0.01 g/L to about 80 g/L, or from about 0.01 g/L to about 90 g/L. In addition to the above mentioned components, the inventive micelle or liposomal formulations can include other drug(s) suitable for use in combination therapy. The inventive compositions also can be stored for extended periods of time, for example, greater than 20 weeks at 25° C. and are believed to exhibit superior systemic delivery and biorelease of their payloads.

As stated above, compositions of the invention can be administered independently or in combination with other therapeutic agents. When used in combination therapy, the inventive compositions can be administered simultaneously with another drug using a single or a separate dosage form or the inventive composition is administered as a separate dosage form within hours or days of the administration of the other therapeutic agent. Examples of compounds/drugs used in such combination therapies include without limitation, chemotherapeutic agents, immunosuppressive agents, immunostimulatory, anti pyretic, cytokines, opioids, cytokines, cytotoxic agents, nucleolytic compounds, radioactive isotopes, receptors, pro-drug activating enzymes, which may be naturally occurring or produced by recombinant methods, anti-inflammatory agents, antibiotics, protease inhibitors, growth factors, osteo-inductive factors and the like.

The inventive formulations can optionally comprise one or more pharmaceutically acceptable excipients, diluents, adjuvants, stabilizers, emulsifiers, preservatives, colorants, buffers, and/or flavor imparting agents, and may be consumed directly or formulated into a nutraceutical or a pharmaceutically acceptable composition suitable for oral, enteral, parenteral, intravenous or topical administration. The inventive formulations can be formulated as a single or a multiple dosage form that is suitable for oral administration. Liquid dosage forms can include, but are not limited to, pharmaceutically acceptable micelles, liposomes, emulsions, solutions, suspensions, syrups and elixirs. The liquid dosage forms may contain inert diluents commonly used in the art. For instance, liquid formulations may contain water, alcohol, polyethylene glycol ethers, glycerol, flavorings, preservatives, essential oils, vitamins or any other pharmaceutically acceptable diluents and/or solvents. Agents used to stabilize emulsions are well known in the pharmaceutical art and such agents can be added to formulations in accordance with the present invention.

For certain formulations an emulsifier such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof may optionally be present. Additionally, oral compositions can include adjuvants such as wetting agents, suspending agents, sweetening, flavoring, and odor imparting agents. When formulated as a suspension, the inventive formulations may contain suspending agents, for example, ethoxylated isostearyl alcohols, polyoxyethylene sorbitol, sorbitan esters, microcrystalline cellulose, aluminum metahydroxide, bentonite, agar-agar, tragacanth, and mixtures thereof. According to one embodiment, micelles containing a mixture of terpene and cannabinoids or a liposome formulation containing a terpene, a hemp oil, a mixture of a cannabinoid and a terpene, or a mixture of hemp oil and a terpene can be formulated as a nutraceutical composition, such as a beverage, drink, or soup that is suitable for oral administration.

The inventive liposomes can be encapsulated by a calcium alginate matrix. A therapeutically effective amount of such an encapsulated liposome formulation of a terpene, hemp oil, mixture of a cannabinoid and a terpene, or a mixture of hemp oil and a cannabinoid can be combined with food or baked goods to obtain a solid nutraceutical composition. Other pharmaceutically acceptable solid dosage forms include without limitation capsules, tablets, pills, powders, and granules of the inventive calcium alginate encompassed liposome formulations of terpene, hemp oil, mixture of cannabinoid and terpene, or a mixture of hemp oil and a cannabinoid.

Pharmaceutically acceptable excipients or a carrier such as sodium citrate or dicalcium phosphate and/or fillers or extenders, such as starches, lactose, sucrose, glucose, mannitol, and silicic acid can also be present in the solid composition. Binders such as carboxymethylcellulose, alginates, gelatin, polyvinylpyrrolidone, sucrose, and acacia; humectants such as glycerol and disintegrating agents such as agar-agar, calcium carbonate, potato or tapioca starch, alginic acid, certain silicates, and sodium carbonate may be present. Solid dosage forms of the calcium alginate encompassed liposome formulation of a terpene, hemp oil, mixture of cannabinoid and terpene, or a mixture of hemp oil and cannabinoid may further contain solution retarding agents such as paraffin; absorption accelerators such as quaternary ammonium compounds; wetting agents such as acetyl alcohol and glycerol monostearate; absorbents such as kaolin and bentonite clay; and lubricants such as talc, calcium stearate, magnesium stearate, solid polyethylene glycols, sodium lauryl sulfate, or mixtures thereof. For capsules, tablets and pills, the dosage form can also comprise buffering agents.

The inventive liposome formulations can be encapsulated with a variety of polymers, sugars, and chelating agents to yield a stable solid liposomal cannabinoid preparation. Encapsulation of the liposomes can take place by cross linking of polymers used as the encapsulating agent. Alternatively, the liposomes can be trapped within a non-crosslinked polymer network, or by dispersing the liposome within the crystalline structure of macromolecules such as sugars, starches or protein molecules. The granules of protein, polymer, sugar or starch encapsulated liposomes can be further processed to yield sublingual films, suppositories, dispersable powder, tablets, or gel capsules.

Solid dosage forms described above may further be coated using compounds that accelerate or decrease the release of the active agents. For instance, the invention encompasses solid dosage forms having enteric coatings, extended-release coatings, sustained-release coatings, delayed release coatings and immediate-release coatings. Methods used to coat solid dosage forms as well as the materials used to manufacture such coatings are well known in the pharmaceutical art. The solid dosage forms may optionally contain opacity enhancing agents.

A dietary composition according to the present invention is any ingestible preparation that contains the suspensions of the invention mixed with a food product. The food product can be dried, cooked, boiled, lyophilized or baked. Breads, teas, soups, cereals, salads, sandwiches, sprouts, vegetables, animal feed, pills and tablets are among the vast number of different food products contemplated in the present invention.

The inventive micelles or liposomes can be formulated as subcutaneous injections, intravenous injections, intramuscular injections, intrasternal injections or infusions for parenteral delivery. Excipients used in the manufacture of parenteral formulations are well known in the art and are non-toxic compounds that do not degrade the active agent or other components of the formulations. Compositions for parenteral injection comprise pharmaceutically acceptable sterile aqueous or non-aqueous solutions, dispersions, suspensions or emulsions as well as sterile powders for reconstitution into sterile injectable solutions or dispersions prior to use. Examples of suitable aqueous and non-aqueous carriers, diluents, solvents or vehicles will include without limitation, water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity will be maintained, for example, by the use of coating materials such as lecithin, by the maintenance of the required particle size in the case of dispersions, and by the use of surfactants. The compositions of the present invention may also contain adjuvants such as, but not limited to, preservatives, wetting agents, emulsifying agents, and dispersing agents. Compositions for parenteral delivery also can include isotonic agents such as sugars, sodium chloride, and the like. Prolonged absorption of the injectable pharmaceutical formulation can be obtained by the inclusion of agents which delay absorption such as aluminum monostearate and gelatin.

Injectable depot forms can be made by microencapsulating the micelle or liposome in a biodegradable polymer such as polylactide-polyglycolide or a polymer of alginic acid. Depending upon the ratio of liposome to polymer or micelle to polymer and the biochemical nature of the polymer employed, the rate of release of a terpene, hemp oil, mixture of cannabinoid and terpene, or a mixture of hemp oil and cannabinoid from the encapsulated micelle or liposome can be controlled. Examples of other biodegradable polymers include poly(orthoesters) and poly(anhydrides). The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Dosage forms for topical administration include, but are not limited to, ointments, creams, lotions, gels and sunscreens comprising a micelle of a terpene and cannabinoid or a liposome containing a terpene, hemp oil, mixture of cannabinoid and terpene, or a mixture of hemp oil and cannabinoid. Such topical formulations can contain agents that promote penetration of the inventive micelles or liposomes through the epidermis. Various other additives known in the art may be included in the topical formulations. Examples of such additives include, but are not limited to, solubilizers, skin permeation enhancers, preservatives (e.g., anti-oxidants), moisturizers, gelling agents, buffering agents, surfactants, emulsifiers, emollients, thickening agents, stabilizers, humectants, dispersing agents and pharmaceutical carriers. Examples of moisturizers include jojoba oil and evening primrose oil. Suitable skin permeation enhancers are well known in the art and include lower alkanols, such as methanol, ethanol and 2-propanol; alkyl methyl sulfoxides such as dimethylsulfoxide (DMSO), decylmethylsulfoxide (C10 MSO) and tetradecylmethyl sulfoxide; pyrrolidones, urea; N,N-diethyl-m-toluamide; C₂-C₆ alkanediols; dimethyl formamide (DMF), N,N-dimethylacetamide (DMA) and tetrahydrofurfuryl alcohol. Examples of solubilizers include, but are not limited to, hydrophilic ethers such as diethylene glycol monoethyl ether (ethoxydiglycol, available commercially as Transcutol®) and diethylene glycol monoethyl ether oleate (available commercially as Softcutol®); polyoxy 35 castor oil, polyoxy 40 hydrogenated castor oil, polyethylene glycol (PEG), particularly low molecular weight PEGs, such as PEG 300 and PEG 400, and polyethylene glycol derivatives such as PEG-8 caprylic/capric glycerides (available commercially as Labrasol®); alkyl methyl sulfoxides, such as DMSO; pyrrolidones, DMA, and mixtures thereof.

One of ordinary skill will appreciate that effective amounts of one or more terpenes, hemp oil, or mixtures of terpene and cannabinoid as well as mixtures of hemp oil and cannabinoid in the compositions can be determined using assays and methodologies known in the art. It is also to be understood that, when administered to a human patient, the total daily usage of the composition of the present invention will be decided by the attending physician within the scope of sound medical judgment. The specific therapeutically effective dose level for any particular patient will depend upon a variety of factors, such as the type and degree of the response to be achieved; the activity of the specific composition employed; the age, body weight, general health, sex and diet of the patient; the duration of the treatment; drugs used in combination or coincidental with the method of the invention; and like factors well known in the medical arts.

The compositions of the invention ca be administered by a variety of other routes, including mucosal, nasal and using transdermal patches. The present invention thus generally described, will be understood more readily by reference to the following examples, which are provided by way of illustration only, and are not intended to be limiting the present invention.

### EXAMPLES

### A. General Protocol for the Manufacture of Emulsions

The manufacture of an emulsion composition comprising a cannabinoid, or a mixture of cannabinoids, hemp oil and one or more terpenes, is prepared by adding an amphipathic molecule such as a phospholipid, including lecithin, sterols, fatty acids to an ethanolic solution, as discussed herein. The ethanolic cannabinoid solution is rapidly injected into distilled water using a Luer Lok syringe or ultrasonic atomizer nozzle. The suspension obtained is then blended with glycerol. A solution of hemp oil, terpenes and a phospholipid are blended into the cannabinoid/ethanol/water/glycerol solution. This gives a homogenous cannabinoid terpene supplement emulsion. The physical properties such as size, composition and concentration of reagents with the emulsion can be controlled by the chemical properties of the terpenes, hemp oil and cannabiniods and by the physical properties of the ethanol/aqueous/glycerol environment used to manufacture the inventive cannabinoid/terpene emulsion supplement.

### B. General Protocol for the Manufacture of Micelles

A micelle composition comprising a cannabinoid, or a mixture of a cannabinoid or its analog and a terpene is prepared by dissolving these components in a water miscible organic solvent followed by the rapid injection of the organic solution into distilled water using a Luer Lock syringe equipped with a 22 gauge needle or an ultrasonic atomizer nozzle. The suspension thus obtained can be concentrated by rotary evaporation to form micro or nano-micellar particles containing cannabinoids or a mixture of a cannabinoid or its analog and a terpene. The size, composition and concentration of the micelles are controlled by the chemical properties of the terpenes and cannabinoids, and by the physical properties of the aqueous-organic solvent used to manufacture the inventive formulations.

### C. General Protocol for the Manufacture of Liposomes

The inventive liposome formulations are manufactured by adding an amphipathic molecule such as a phospholipid, including lecithin, sterols, or fatty acids, to an organic solution of a terpene, hemp oil, cannabinoid or a cannabinoid analog, a mixture of a cannabinoid or a cannabinoid analog and a terpene, or a mixture of a cannabinoid or a cannabinoid analog and hemp oil. This solution is rapidly injected into distilled water using (a) a Luer Lock syringe equipped with a 22 gauge needle or (b) an ultrasonic atomizer nozzle, and the suspension thus obtained is concentrated by rotary evaporation to obtain liposomes containing a terpene, hemp oil, cannabinoid or a cannabinoid analog, a mixture of a cannabinoid or a cannabinoid analog and a terpene, or a mixture of a cannabinoid or a cannabinoid analog and hemp oil. The physical properties such as size, composition and concentration of reagents within the liposome can be controlled by the chemical properties of the terpenes, hemp oil and cannabinoids, and by the physical properties of the organic solvent/aqueous environment used to manufacture the inventive liposome formulation.

In an exemplary composition, the hydrophobic/lipophilic membrane of the liposome can comprise about 40% phosphatidylcholine, about 3.5% phosphatidylethanolamine, about 6% phosphonophospholipids, and about 0.5% of other phospholipids. According to another exemplary composition the hydrophobic/lipophilic membrane of liposomes in the inventive composition can comprise about 26% phosphatidylcholine, about 10% phosphatidylethanolamine, about 13% phosphonophospholipids, and about 1% of other phospholipids.

### D. General Protocol for Encapsulation of Liposomes

The present invention also provides a method for encapsulating the inventive liposome suspension. Pursuant to this method, a polymer or encapsulating matrix is added at a desired concentration to the liposome composition prepared as described above. A suitable cross linking agent can then be added to the polymer-liposome reaction mixture to initiate cross-linking. After cross-linking the polymer or encapsulating matrix, the reaction will be dehydrated by rotary evaporation, freeze drying, or spray drying to obtain the encapsulated liposome compositions. The dehydrated encapsulated compositions can be milled to obtain a powder having a desired average particle size.

The present invention uses micelles of a terpene and a cannabinoid or liposomes as vehicles for the delivery of formulations containing a terpene, hemp oil, cannabinoid or a cannabinoid analog, a mixture of terpenes and a cannabinoids or a cannabinoid analog, or a mixture of hemp oil and a cannabinoid or a cannabinoid analog to a subject in need thereof. The micelles and liposomes of the invention are dispersed in a pharmaceutically acceptable solvent that is suitable for a specific route of delivery to a subject in need of treatment.

### I. Emulsion Formulation

The formulations of the present invention may comprise hemp oil, cannabinoids and terpenes or any mixture thereof. This emulsion is prepared by first dissolving cannabinoids into ethanol. Using a 60 mL sterile syringe with a 16 gauge needle, 33 ml of the cannabinoid-ethanol solution is injected into 90 mL sterile distilled water, contained in a 500 ml Sterile Media bottle. The mixture is blended using a Polytron Blender on Low Speed for 30 seconds. Using a syringe, 90 mL of glycerin is injected into the solution. The mixture is blended once more using the Polytron Blender on Low Speed for 30 seconds. 3.3 g lecithin is blended into 90 ml hemp oil at room temperature. To this solution 7.5 ml of a terpene formulation is added. The mixture is homogenized using the Polytron Blender on Low Speed for 30 sec. The lecithin-hemp oil-terpene solution is added to the cannabinoid-EtOH-Water-glycerol mixture. It is homogenized using a Polytron Blender on Low Speed for 30 seconds to create an homogenous emulsion.

### II. Terpene Formulations

The formulations of the present invention may contain one or more terpenes. In certain embodiments, the formulations contain a mixture of terpenes. This mixture contains a primary terpene in an amount of up to 50%, a secondary terpene in an amount from about 30% to about 40%, and a tertiary terpene in an amount from about 8% to about 10%. The formulation also may optionally contain trace amounts (0-5%) of other terpenes. The category of “terpene formulations” includes eight groups and several subgroups as further described below:

### Group I: Primary Terpene is α-Pinene

### Subgroup A

### Secondary: Myrcene

Tertiary: β-Pinene and t-caryophyllene

### Trace: α-Humulene, α-Bisabolol, Guaiene, Limonene, Ocimene, Terpinolene, 3-Carene

### Subgroup B

Secondary: β-pinene,
Tertiary: t-Caryophyllene, β-Bisabolol,
Trace: myercene, Guaiol, α-Terpineol, Limonene, Linalool

### Subgroup C

Secondary: t-Caryophyllene,
Tertiary: myrcene, β-pinene

### Trace: α-Bisabolol, Guaiol, Limonene

### Group II. Primary Terpene is α-Bisabolol

Secondary: t-Caryophyllene

### Tertiary: α-Pinene and Myrcene

Trace: Guaiol, Linalool, α-Humulene, alpha-terpineol

### Group III. Primary Terpene is β-Pinene

Secondary: α-pinene
Tertiary: t-Caryophyllene and Terpinolene

### Trace: Myrcene

### Group IV. Primary Terpene is Guaiene

Secondary: t-Caryophyllene

### Tertiary: Myrcene and α-Humulene

### Trace: α-Pinene, α-Bisabolol, β-Pinene, Limonene, Ocimene, Terpinolene

### Group V. Primary Terpene is Guaiol

### Secondary: α-Bisabolol

Tertiary: t-Caryophyllen and Myrcene

### Trace: α-Pinene, α-Terpineol, α-Humulene, Terpinolene

### Group VI. Primary Terpene is Limonene

### Subgroup A

Secondary: t-Caryophyllene

### Tertiary: Linalool, Myrcene

### Trace: α-Humulene, α-Pinene, α-Terpineol, β-Pinene, Fenchol, Guaiene

### Subgroup B

### Secondary: Myrcene

Tertiary: β-pinene and t-Caryophyllene

### Trace: α-Pinene, Guaiene, Linalool, Ocimene

### Group VII. Primary Terpene is Myrcene

### Subgroup A

### Secondary: α-Pinene

Tertiary: β-pinene and t-Caryophyllene

### Trace: 3-Carene, α-Bisabolol, Guaiene, Guaiol, Limonene, Linalool, Ocimene, Terpinolene

### Subgroup B

Secondary: t-Caryophyllene

### Tertiary: Limonene, α-Pinene

### Trace: α-Humulene, α-Bisabolol, β-Pinene, Guaiene, Guaiol, Limonene, Linalool, Ocimene,

### Terpinolene

### Subgroup C

### Secondary: Terpinolene

Tertiary: t-Caryophyllene and Ocimene

### Trace: α-Pinene, β-Pinene, Guaiol, Limonene

### Subgroup D Secondary: Ocimene

Tertiary: t-Caryophyllene and α-Pinene

### Trace: α-Pinene, β-Pinene, Limonene, Terpinolene

### Subgroup E

### Secondary: Limonene

Tertiary: t-Caryophyllene

### Trace: α-Pinene, β-Pinene, Linalool, Ocimene

### Subgroup F

### Secondary: Linalool

Tertiary: t-Caryophyllene

### Trace: α-Terpineol, β-Pinene, Limonene

### Group VIII. Primary Terpene is Ocimene

Secondary: t-Caryophyllene

### Tertiary: Myrcene and Limonene

### Trace: 3-Carene, α-Pinene, β-Pinene, Terpinolene

### III. Micelle Terpene/Cannabinoid Formulations

### Example 1

### Micelle Suspension of Terpenes and Cannabinoids without Stabilizer

Micelle formulations in accordance with the present invention containing terpenes and cannabinoids in amounts described above can be prepared by dissolving 750-1500 mg of terpenes and a cannabinoid extract containing THC, CBC, CBD or mixtures of these cannabinoids or one or more analogs of a naturally occurring cannabinoid or a Formula I cannabinoid in 95% ethanol and the volume of this solution is brought to 20 ml using 95% EtOH. The ethanolic solution containing terpenes and cannabinoids is cooled to 10° C. prior to injecting this solution into 195 ml of distilled water (25° C.), at a pressure of 50 psi and a flow rate of 10 mL/min using a 50 mL Luer Lock syringe equipped with a 22 gauge needle. The resultant solution is rotary evaporated under a reduced pressure to remove ethanol and provide an aqueous micelle composition containing a mixture of terpenes and cannabinoids.

### Example 2

### Micelle Suspension of Terpenes and Cannabinoids with Stabilizer

The protocol for the manufacture of a stabilized micelle suspension containing a mixture of terpenes and cannabinoids in amounts described above is similar to the one described above. Briefly, 750-1500 mg of one or more terpenes and a cannabinoid extract are dissolved in 95% ethanol. The final volume of this solution is brought to 20 ml with 95% EtOH. After cooling to 10° C. the ethanolic solution is injected at a pressure of 50 psi and a flow rate of 10 ml/min into 195 ml of distilled water (25° C.), using a 50 mL Luer Lock syringe equipped with a 22 gauge needle. The resultant solution is concentrated using a rotary evaporator to remove ethanol and 0.2 g, (0.1% w/v) guar gum is added in 10 mg portions (0.2 g), to the concentrated solution to obtain a stabilized micelle composition comprising a mixture of terpenes and cannabinoids.

### IV. Liposomal Terpene Formulations

### Example 3

### Liposomal Suspensions of Terpenes

15 g of terpene was dissolved in 95% ethanol and the final volume of this solution was brought to 30 ml with 95% EtOH. To this ethanolic solution of terpene was added 30 ml of an ethanolic solution of lecithin-50 which was prepared by dissolving 15 grams of lecithin-50 in 95% EtOH and bringing the volume of the lipid/EtOH solution to 30 ml by the addition of 95% EtOH. After cooling to 10° C. the ethanolic lipid/terpene solution was injected at a pressure of 50 psi and a flow rate of 10 mL/min, into 540 ml of distilled water (25° C.), using a 100 mL Luer Lock syringe equipped with a 22 gauge needle.

Alternatively, the ethanolic solution of the lipid and terpene at 30° C. was injected into distilled water through a 0.17 mm stainless steel orifice at 300 psi. According to yet another embodiment, an ethanolic solution of the lipid and terpene at 25° C. was injected into 1.2 L of distilled water (25° C.), using an Ultrasonic Atomizer Nozzle at 60 Hz. The size of the drops injected into water were about 20 μm and the lipid and terpene was injected at a flow rate of 10 mL/min. The ethanolic/aqueous suspension thus obtained was concentrated to a volume of about 200 mL by rotary evaporation keeping the temperature of the solution below 55° C.

The final maximum concentration of terpene in the liposomes obtained using the above described method was 70 g/L and the aqueous liposomal suspension was stable for more than 3 months at 25° C. Moreover, the average diameter of the liposomes was in the range from about 200 nm about 400 nm. Oil immersion microscopic analysis showed the liposomes to have an aqueous core.

### Example 4

### Liposomal Suspensions of Terpenes and Cannabinoids

Liposome formulations in accordance with the present invention containing terpenes and cannabinoids in amounts described above can be prepared by dissolving 15 g of terpenes and an extract comprising one or more cannabinoids (e.g., THC, CBC, CBD, their mixtures or analogs) in 95% ethanol and the final volume of this solution is brought to 30 ml using 95% EtOH. To the solution of terpene and cannabinoids is added an ethanolic solution of lecithin-50 (30 ml) that is separately prepared by dissolving 15 grams of lecithin-50 into 95% EtOH (30 ml). After cooling to 10° C. the ethanolic solution containing a mixture of lipid, terpene and cannabinoid is injected into 540 ml of distilled water (25° C.), at a pressure of 50 psi and a flow rate of 10 mL/min using a 100 mL Luer Lock syringe equipped with a 22 gauge needle to obtain an aqueous ethanolic liposome formulation containing terpene and cannabinoid.

Liposomes containing a mixture of a terpene and a cannabinoid also can be prepared by injecting an ethanolic solution (30° C.) containing the mixture of lipid, terpene and cannabinoid into distilled water using a 0.17 mm stainless steel orifice and an injection pressure of 300 psi. Alternatively, an Ultrasonic Atomizer Nozzle (60 Hz, 20 μm drop size, flow rate 10 mL/min) can be used to inject the ethanolic solution of lipid, terpene and cannabinoid into 1.2 L of distilled water. The aqueous ethanolic liposomal formulation produced pursuant to any of the above described protocols can be concentrated to a volume of 200 mL using rotary evaporation while maintaining the temperature of the solution below 55° C. to obtain an aqueous liposome formulation containing a mixture of a terpene and a cannabinoid.

The liposome formulations pursuant to any of the above methodologies should contain a mixture of terpene and cannabinoid. The concentration of terpene in such a formulation can be in the range of about 0.001 g/L to about 100 g/L, while the cannabinoid concentration can be from about 0.1 g/L to about 100 g/L. The aqueous liposomal suspension, moreover, should be stable for more than 3 months at 25° C. and the average size of liposomes in such a formulation should be in the range from about 200 nm to about 400 nm in diameter.

### V. Encapsulation Formulations

### Example 5

### Calcium Alginate Encapsulation of Liposomal Suspensions

Encapsulated liposome formulations in accordance with the present invention containing terpenes and cannabinoids in amounts described above can be prepared by adding 4 g sodium alginate to 200 ml of an aqueous liposomal formulation of terpenes or a liposomal suspension of a terpene/cannabinoid mixture obtained using the protocols described above in Examples 3 and 4.

To promote encapsulation, the aqueous formulation of alginate and liposome is poured into 40 mL of a stifling aqueous solution of calcium chloride (25 wt %) and the entire reaction mixture is allowed to stir for an additional 10 minutes to permit complete crosslinking of the aliginic acid. The solid mass of calcium alginate-encapsulated liposomes is then cold pressed to remove water and air-dried for 24 hours in warm air at 50° C. The air-dried material is milled to obtain a free flowing yellow-white powder that can be dissolved in buffer. Encapsulated liposomes obtained using the above described methodology permit complete release of terpene or the mixture of terpene and cannabinoid in 60 mM citrate buffer (pH 7).

### Example 6

### Film Formation of Liposomal Suspensions

A film of the inventive liposome formulation containing a final concentration of terpenes and cannabinoids in amounts described above is prepared by adding sodium alginate to the liposomal suspensions prepared in Examples 3 and 4 above to obtain a composition having a final alginate concentration of 4% w/v (8 grams). This solution is stirred at room temperature to dissolve the alginate and then poured into a flat tray to form a layer having a depth of about 0.5 cm. An aqueous solution of calcium chloride is then mixed with the aqueous layer containing alginate and liposome and the resultant mixture is permitted to stand at room temperature to promote the formation of a film through crosslinking of the alginic acid.

The film thus formed is pressed to remove water and dried for 24 hours using warm air at 50° C. The dried film, about 1 mm in thickness, can be cut into readily consumable strips or milled to a free flowing powder. The films obtained using the above described methodology should permit complete release of terpene or the mixture of terpene and cannabinoid in distilled water.

### Example 7

### Formation of a Dispersible Dry Powder

The liposomal preparations obtained in Examples 3 and 4 can be diluted 1:10 with distilled water. To 200 ml of the diluted solution is added 40 grams of lactose or sucrose (200 mg/ml) and 0.12 grams L-leucine (0.6 mg/ml). Following dissolution of the added components, the entire suspension is poured into a flat tray to form a layer having a depth of about 0.5 cm. This layer is allowed to set to form a gel like solid which can be used directly. Alternatively, the gel is compressed to remove water and the resultant solid can be further dried at room temperature or spray-dried with a forced air spray dryer at a temperature of 55° C. to obtain a crystalline solid that can be milled to a free flowing powder. The powder thus obtained should dissolve completely in water and release greater than 90% of the liposomes. The amount of cannabinoid or cannabinoid analogue in the aqueous terpene/cannabinoid solution is from about 10% w/w to about 80% w/w and the amount of terpene in the aqueous terpene-cannabinoid solution is from about 1% w/w to about 10% w/w.

### VI. Liposome Formulation

### Example 8

### Liposomal Suspensions of Hemp Oil

Liposome formulations in accordance with the present invention containing hemp oil in amounts described above can be prepared by dissolving 15 g of Hemp oil in 95% ethanol and the final volume of this solution is brought to 30 ml using 95% EtOH. In a separate flask, 15 grams of lecithin-50 is dissolved in 30 ml of 95% EtOH and this solution of lecithin-50 is added to the solution of hemp oil to obtain a lecithin-hemp oil mixture. After cooling the lecithin-hemp oil mixture to 10° C., the cold solution is injected into 540 ml of distilled water at a pressure of 50 psi and a flow rate of 10 ml/min, using a 100 ml Luer Lock syringe equipped with a 22 gauge needle to obtain an aqueous-ethanolic liposome formulation of hemp oil.

Alternatively, liposomes containing hemp oil are obtained by introducing a 30° C. ethanol solution of hemp oil into distilled water using a 0.17 mm stainless steel orifice at a pressure of 300 psi. An Ultrasonic Atomizer Nozzle (60 Hz, 20 μm drop size, and a flow rate of 10 ml/min), also can be used to introduce the ethanolic solution of hemp oil into distilled water (1.2 L). The resultant aqueous alcoholic liposome suspension is concentrated by rotary evaporation keeping the temperature of the solution in the flask below 55° C. to obtain an aqueous suspension of liposomes containing hemp oil.

The final maximum concentration of hemp oil in an aqueous suspension of liposomes prepared using the above described methodology is in the range from about 0.01 g/L (0.01 mg/mL) to about 200 g/L (200 mg/mL) and liposomes formulations of hemp oil obtained using the protocol described above should be stable for more than 3 months at 25° C.

### Example 9

### Liposomal Suspensions of Hemp Oil and Cannabinoids

A protocol similar to the one described above in Example 8 can be used for the manufacture of liposome containing a mixture of hemp oil and cannabinoids. Such formulations should contain hemp oil in an amount from about 0.001 g/L to 200 g/L and cannabinoids in an amount from about 0.01 g/L to about 100 g/L as described above. Briefly, a mixture of hemp oil (15 g) and one or more cannabinoids or a cannabinoid analog obtained by contacting a Formula I compound with a cannabinoid synthase is dissolved in 30 mL of 95% ethanol. This ethanolic hemp oil-cannabinoid solution is combined with 30 ml of a 95% ethanol solution of lecithin-50 prepared according to the protocol described above in Example 8. The lecithin-hemp oil/cannabinoid solution is cooled to 10° C. prior to injecting the cold solution (pressure—50 psi and flow rate—10 ml/min), into 540 ml of distilled water using a 100 ml Luer Lock syringe equipped with a 22 gauge needle. The resultant mixture is an aqueous-ethanolic suspension of liposomes containing a mixture of hemp oil and cannabinoids or hemp oil and a cannabinoid analog.

Alternatively, liposomes containing a mixture of hemp oil and cannabinoids or a cannabinoid analog can be obtained by introducing a 30° C. ethanol solution of a mixture of hemp oil and cannabinoids or hemp oil and a cannabinoid analog into distilled water using a 0.17 mm stainless steel orifice at a pressure of 300 psi. An Ultrasonic Atomizer Nozzle (60 Hz, 20 μm drop size, and a flow rate of 10 ml/min) also can be used to introduce the ethanolic solution containing a mixture of hemp oil and cannabinoids or a cannabinoid analog into distilled water (1.2 L). The resultant aqueous alcoholic liposome suspension is then concentrated by rotary evaporation, keeping the temperature of the solution within the flask below 55° C., to obtain an aqueous suspension of liposomes containing a mixture of hemp oil and cannabinoids or a cannabinoid analog. The liposome suspension obtained using the methods described above should be stable for more than 3 months at 25° C.

### VII. Encapsulation Formulations

### Example 10

### Calcium Alginate Encapsulation of Liposomal Suspensions

Sodium alginate (4 g) is dissolved in 200 ml of the hemp oil liposomal suspension prepared in Example 8 or the liposomal suspension of hemp oil and cannabinoid according to Example 9. The resultant mixture is poured into 40 ml of an aqueous solution of 25% calcium chloride to initiate crosslinking of alginic acid and promote the encapsulation of liposomes. The solid mass of calcium alginate encapsulated liposomes thus obtained are cold-pressed to remove excess water, followed by air drying at 50° C. for 24 hours. The air-dried material is milled to a free flowing powder that is dissolved in buffered water prior to use.

The amount of hemp oil in the alginate powder as described above is from about 10% w/w to about 80% w/w. When a liposome formulation containing a mixture of cannabinoid and hemp oil is used for encapsulation, the cannabinoid content in the alginate powder is from about 10% to about 80% while the amount of hemp oil is from about 90% to about 20%. The powder should completely release the encapsulated components when contacted with 60 mM citrate buffer at pH 7.

### Example 11

### Film Formation of Liposomal Suspensions

Films of liposome suspensions are obtained by adding sodium alginate to the liposome suspensions manufactured using the protocol described in Examples 8 and 9. The final concentration of sodium alginate in the liposome suspensions is 4% w/v (8 grams). The resultant solution is stirred at room temperature to dissolve the alginate and then poured into a flat tray to form a layer having a depth of about 0.5 cm. Calcium chloride is added to the solution in the tray to crosslink the alginic acid-liposome mixture and form a gel like solid. This gel like solid can be desiccated in a drying cabinet at a temperature of 50° C. to obtain a 1 mm thick film. The final film should contain approximately 10% to 80% hemp oil or a mixture of hemp oil and cannabinoids, and should completely dissolve in distilled water.

### Example 12

### Formation of a Dispersible Dry Powder

Liposomal compositions according to Examples 8 and 9 are diluted 1:10 with distilled water. 40 grams maltodextrin, lactose or sucrose (200 mg/ml) and 0.12 grams L-leucine (0.6 mg/ml) are added to 200 ml of each the liposome compositions. The resultant solutions are frozen using a dry ice/acetone bath and then freeze dried to a powder. Alternatively, the liposome solutions containing sugar and leucine can be spray-dried using a forced air spray dryer at a temperature of 55° C. to obtain a crystalline solid than can be milled to obtain a free flowing powder.

The dry powder thus obtained should contain approximately 10% to 80% hemp oil or a mixture of hemp oil and a cannabinoid and should dissolve completely in water and release greater than 90% of the starting liposomes into solution.

The scope of the claims should not be limited by the preferred embodiments set forth in the examples, but should be given the broadest interpretation consistent with the description as a whole.

1. A stable, aqueous liposome formulation of a terpene comprising:
a primary terpene selected from the group consisting of α-pinene, α-bisabolol, β-pinene, guaiene, guaiol, limonene, myrcene and ocimene;
a secondary
terpene; and a
tertiary terpene,
wherein the amount of the primary terpene is 50% (w/w), the amount of the secondary terpene is from about 30% to about 40% (w/w) and the amount of the tertiary pinene is from about 8% to about 10% (w/w) of the formulation.

2. The formulation according to claim 1, further comprising one or more cannabinoids or cannabinoid analogues, wherein the average diameter of the liposome in the formulation is in a range between 50 nm and 1000 nm.

3. The formulation according to claim 2, wherein the final maximum concentration of cannabinoids or cannabinoid analogues is from about 0.01 g/L to about 100 g/L.

4. (canceled)

5. (canceled)

6. The formulation according to claim 2, wherein the one or more cannabinoids or cannabinoid analogues are selected from the group consisting of cannabinol, cannabidiol, Δ9-tetrahydrocannabinol, Δ8-tetrahydrocannabinol, 11-hydroxy-tetrahydrocannabinol, 11-hydroxy-Δ9-tetrahydrocannabinol, levonantradol, Δ11-tetrahydrocannabinol, tetrahydrocannabivarin, dronabinol, amandamide and nabilone and a combination of two or more of these compounds.

7. The formulation according to claim 2, further comprising a stabilizer selected from the group consisting of guar gum, xyanthan gum cellulose hyaluronic acid, polyvinyl pyrrolidone (PVP), alginate, chondritin sulfate, poly gamma glutamic acid, gelatin, chitisin, corn starch and flour, in an amount from about 0.1% to about 2% (w/v).

8. The formulation according to claim 1, wherein the primary terpene is α-pinene; the secondary terpene is selected from the group consisting of myrcene, β-pinene and t-carophyllene; the tertiary terpene is selected from the group consisting of β-pinene, t-carophyllene, α-bisabolol and myrcene, and the suspension further comprises trace amounts of one or more terpenes selected from the group consisting of α-humulene, α-bisabolol, guaiene, limonene, ocimene, terpinolene, 3-carene, myercene, guaiol, α-terpineol and linalool.

9. The formulation according to claim 1, wherein the primary terpene is α-bisabolol; the secondary terpene is t-carophyllene; the tertiary terpene is selected from the group consisting of α-pinene and myrcene, and the suspension further comprises trace amounts of one or more terpenes selected from the group consisting of α-humulene, α-terpineol guaiol, and linalool.

10. The formulation according to claim 1, wherein the primary terpene is β-pinene; the secondary terpene is α-pinene; the tertiary terpene is selected from the group consisting of t-carophyllene and terpinolene; and the suspension further comprises trace amounts of myrcene.

11. The formulation according to claim 1, wherein the primary terpene is guaiene; the secondary terpene is t-carophyllene; the tertiary terpene is selected from the group consisting of myrcene and α-humulene; and the suspension further comprises trace amounts of α-pinene, bisabolol, β-pinene, limonene, ocimene and terpinolene.

12. The formulation according to claim 1, wherein the primary terpene is guaiol; the secondary terpene is α-bisabolol; the tertiary terpene is selected from the group consisting of t-carophyllene myrcene; and the suspension further comprises trace amounts of α-pinene, α-terpineol, α-humulene and terpinolene.

13. The formulation according to claim 1, wherein the primary terpene is limonene; the secondary terpene is selected from the group consisting of myrcene and t-carophyllene; the tertiary terpene is selected from the group consisting of linalool, myrcene, β-pinene and t-carophyllene, α-bisabolol and myrcene, and the suspension further comprises trace amounts of one or more terpenes selected from the group consisting of α-humulene, α-pinene, β-pinene, fenchol, guaiene, linalool, ocimene and α-terpineol.

14. The formulation according to claim 1, wherein the primary terpene is myrcene; the secondary terpene is selected from the group consisting of α-pinene, t-carophyllene, terpinolene, ocimene, limonene and linalool; the tertiary terpene is selected from the group consisting of β-pinene, t-carophyllene, limonene, ocimene and myrcene; α-pinene, bisabolol and myrcene, and the suspension further comprises trace amounts of one or more terpenes selected from the group consisting of α-humulene, α-bisabolol, guaiene, limonene, ocimene, 3-carene,
β-pinene, α-pinene, myercene, guaiol, α-terpineol, terpinolene and linalool.

15. (canceled)

16. (canceled)

17. (canceled)

18. (canceled)

19. (canceled)

20. (canceled)

21. A method of producing a stable liposomal formulation of one or more terpenes comprising the steps of:
(a) dissolving one or more terpenes in ethanol to obtain an ethanolic solution of terpenes;
(b) adding a phospholipid to the ethanolic solution of terpenes;
(c) injecting the solution from step (b) into distilled water to obtain an aqueous alcoholic liposomal formulation of terpenes; and
(d) removing the ethanol from the aqueous ethanolic liposome formulation of terpenes, thereby producing a stable aqueous liposomal formulation of one or more terpenes;
wherein the final maximum concentration of terpenes in the liposomal formulation is from about 0.001 g/L to about 100 g/L

22. The method of claim 21, wherein the final maximum concentration of terpenes in the liposomal formulation is from about 10 g/L to about 70 g/L.

23. The method of claim 21, wherein step (a) further comprises dissolving one or more cannabinoids or cannabinoid analogues selected from the group consisting of cannabinol, cannabidiol, Δ9-tetrahydrocannabinol, Δ8-tetrahydrocannabinol, 11-hydroxy-tetrahydrocannabinol, 11-hydroxy-Δ9-tetrahydrocannabinol, levonantradol, Δ11-tetrahydrocannabinol, tetrahydrocannabivarin, dronabinol, amandamide, nabilone, and a combination thereof.

24. The method of claim 23, wherein the average diameter of the liposome is in a range between 50 nm and 1000 nm, and wherein the final maximum concentration of cannabinoids or cannabinoid analogues in the suspension is from about 0.01 g/L to about 100 g/L.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. An aqueous solution of a terpene and a cannabinoid comprising a primary terpene, a secondary terpene, a tertiary terpene, and one or more cannabinoids or cannabinoid analogues, wherein the total amount of the terpene and cannabinoid or terpene and cannabinoid analog is 50 g/liter.

40. The aqueous solution according to claim 38, wherein the solution is in form of a fast-acting pharmaceutical composition, a nutraceutical composition, or a food or beverage for administration to a subject.

41. The aqueous according to claim 40, wherein the pharmaceutical composition and the nutraceutical composition are fast-acting formulations for oral, enteral, parenteral, intravenous, pulmonary, mucosal, sub-mucosal or topical administration.

42. (canceled)

43. (canceled)

44. (canceled)
